(19) United States
(12) Patent Application Publication
(10) Pub. No.: US 2021/0085913 A1
(43) Pub. Date: Mar. 25, 2021

(54) GARMENT WITH ATTACHED ORNAMENT

(71) Applicants: Brigitte Bastaldo-Tsampalis, Bridgewater, NJ (US); Amalia Kylie Tsampalis, Bridgewater, NJ (US); Beatrix Nefeli Tsampalis, Bridgewater, NJ (US)

(72) Inventors: Brigitte Bastaldo-Tsampalis, Bridgewater, NJ (US); Amalia Kylie Tsampalis, Bridgewater, NJ (US); Beatrix Nefeli Tsampalis, Bridgewater, NJ (US)

(21) Appl. No.: 16/579,164

(22) Filed: Sep. 23, 2019

(51) Int. Cl. A61M 21/02; A41D 27/08

(52) U.S. Cl. CPC A61M 21/02; A61M 2021/0016; A41D 27/08

(57) ABSTRACT

Systems and methods of removably providing a functional ornament on a garment. The garment, which may be a shirt, includes a body configured to cover at least a portion of a center portion of an individual wearing the garment. The body has openings configured to receive different appendages of the individual passing therethrough. An anchor segment is attached to the body such that the anchor segment lies substantially flat against the body adjacent to the anchor segment. The ornament, which includes at least one of an aromatic compound, a crystal, a medicinal doser or a wellness insight technology is replaceably attached to the anchor segment.

### TECHNICAL FIELD

Embodiments pertain to garments. Some embodiments pertain to garments with functional ornaments attached thereto.

### BACKGROUND

Apparel and garments come in many types and styles, covering different portions of the body. Upper body apparel, for example, includes more formal collared shirts, semiformal V-neck shirts and sweaters, and more informal shirts, such as t-shirts and tank tops, among others. In many cases, there is a desire to individualize one's garments. Typically, such individualization is done at the time of purchase—i.e., a garment is purchased in the desired style. However, this may be constraining, as well as costly, as multiple garments may be used to fulfill various desires.

### BRIEF DESCRIPTION OF THE FIGURES

In the figures, which are not necessarily drawn to scale, like numerals may describe similar components in different views. Like numerals having different letter suffixes may represent different instances of similar components. The figures illustrate generally, by way of example, but not by way of limitation, various aspects discussed in the present document.

FIG. 1 illustrates a plan view of a garment in accordance with some embodiments.

FIG. 2 illustrates a close up of a portion of the garment shown in FIG. 1.

FIG. 3 illustrates electronic components of an ornament in accordance with some embodiments.

### DETAILED DESCRIPTION

The following description and the drawings sufficiently illustrate specific aspects to enable those skilled in the art to practice them. Other aspects may incorporate structural, logical, electrical, process, and other changes. Portions and features of some aspects may be included in, or substituted for, those of other aspects. Aspects set forth in the claims encompass all available equivalents of those claims.

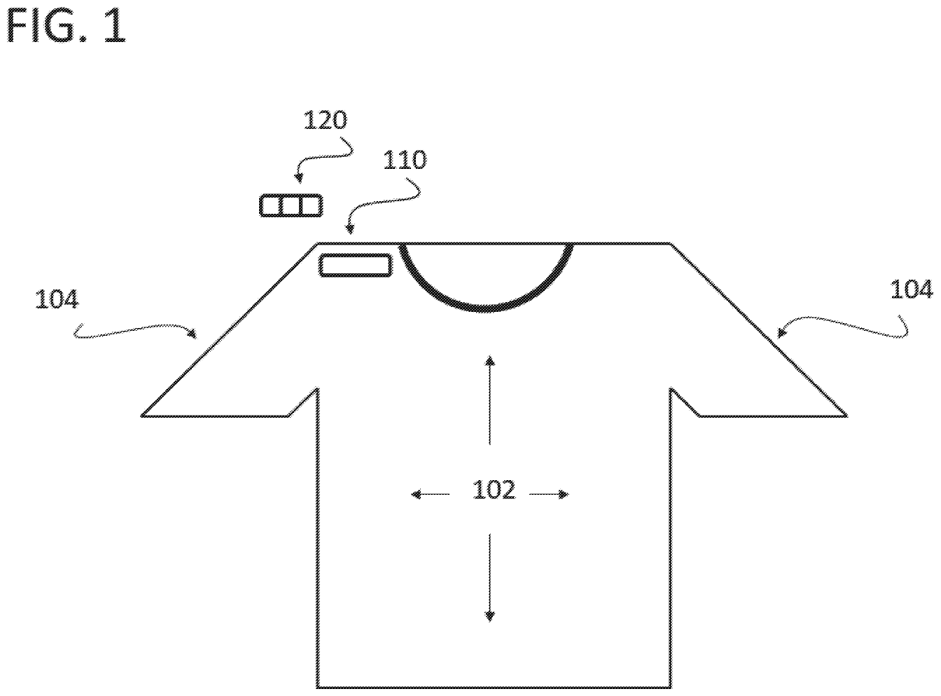

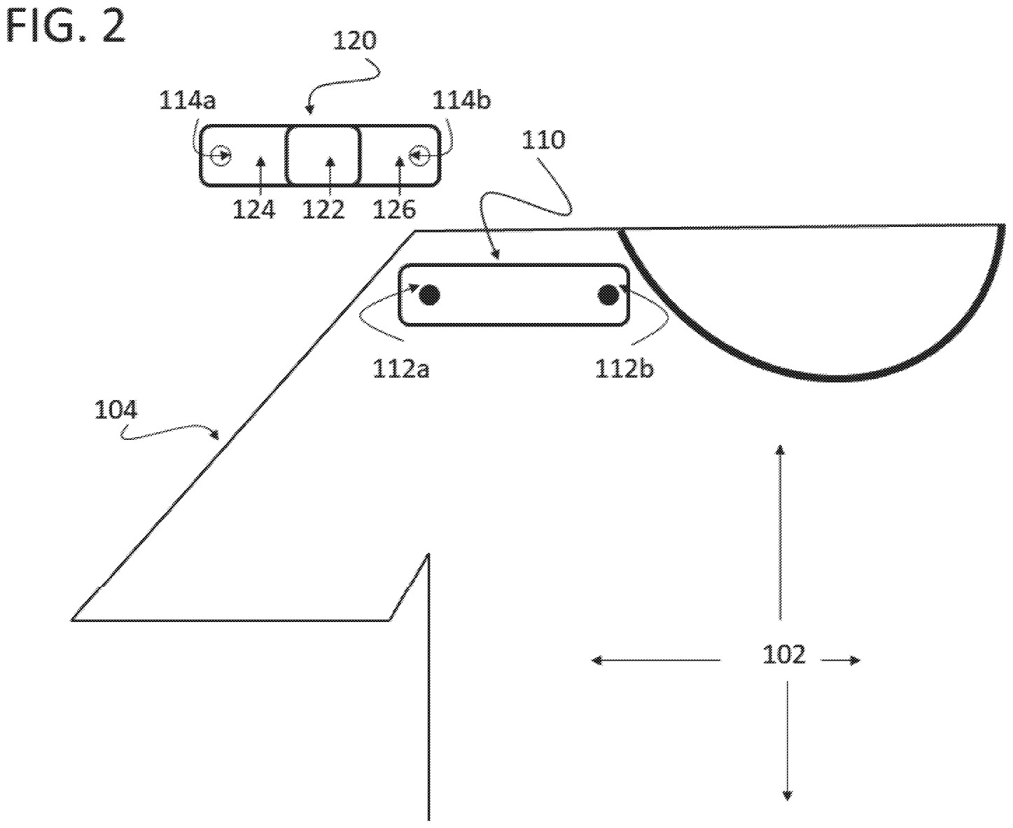

FIGS. 1 and 2 show plain and close up views of the garment 100. The garment 100 may be a shirt, such as a t-shirt as shown. In other embodiments, the garment 100 may be a shirt such as a collared shirt, sweater, hoodie, cardigan, tank, athletic bra like apparel or any other top that may be worn without, or with, an overlying coat or jacket.

As shown, the shirt 100 may have a body that comprises a main portion 102 and sleeves 104. The main portion 102 is configured to cover at least a portion of the torso of an individual wearing the shirt 100, while the sleeves 104 are configured to cover at least a portion of the arms of the individual. The main portion 102 may have openings for the head and torso of the individual to extend therethrough. The sleeves 104 may have openings for the arms of the individual to extend therethrough. The body may be formed from any desired shirt fabric, e.g., a lightweight, breathable fabric can be used for hot weather situations, a medium weight fabric for cool weather, and a heavyweight, fleece fabric for cold weather. The body may be formed for any age and size, suited for any individual regardless of their mobility capabilities or physical disabilities, inclusive of the missing or injured appendages or body parts.

The shirt 100 may have an anchor segment 110 attached to the shoulder portion of main portion 102 or sleeve 104 of the body such that the anchor segment 110 lies substantially flat against the body adjacent to the anchor segment 110. As shown, the anchor segment 110 is attached in some embodiments to a shoulder portion of the main portion 102, at or near the apex of the shoulder portion. In other embodiments, the anchor segment 110 may be attached to sleeve portion as far down as the wrist section of an individual. In some embodiments, each anchor may be a continuous piece or segmented, consisting of pieces 112a and 112b that may be simply a snap in the fabric forming the shirt 100. In other embodiments, the anchor segment 110 may be formed from a fabric. The fabric forming the anchor segment 110 may be the same as that forming the shirt 100 or may be different from that forming the shirt 100. In the latter case, the fabric forming the anchor segment 110 may have a similar color as fabric of the shirt 100 the local area in which the anchor segment 110 is attached to the shirt 100, or the colors of the fabric forming the anchor segment 110 and the fabric forming the shirt 100 may be different in the local area. The color of the fabric forming the anchor segment 110 may be different from that of the entire body or may be the same as another portion of the shirt 100.

In some embodiments, the anchor segment 110 may be permanently attached to the body, e.g., via sewing. The anchor segment 110 may be attached as to the body at opposite ends of the anchor segment 110. The anchor segment 110 may include pairs of anchor segment pieces 112a, 112b on opposite sides of the ornament 120. Each anchor segment piece 112a, 112b may be attached at an end of the anchor segment piece 112a, 112b most distal from the ornament 120. In other embodiments, each anchor segment piece 112a, 112b may be removably attached to the body, e.g., by Velcro, snap button and all associated embodiment, hooks and all associated embodiment, tradition slip buttons, clasps, semi-permanent glue, magnetic attachment pieces and buckles.

As shown, an ornament 120 may be replaceable attached to the anchor segment 110. The ornament 120 may have a body 122, support 124 and attachment mechanism 126. The body of the ornament 120 may be formed from one or more materials such as plastic, resin, silicon, metal, textile, stone, ceramics etc. The ability to manually remove and replace (change) or reattach the same ornament may permit selection of the desired ornament, as well as allow the garment 100 to be cleaned without damaging the ornament (which may be removed from the garment before cleaning). The ornament 120 may be an active ornament that contains a functional insert, such as an aromatherapy insert that permits an aromatic compound to be released therefrom. The ornament 120 may in addition, or instead, itself be a healing crystal or geode.

In some embodiments, the ornament 120 may function in the provision of medicinal dosing, acting as a medicinal doser, or may itself include one or more wellness insights technologies each of which may be capable of collecting, displaying or communicating. Medicinal dosing functionality may include, among others, tactile electrodes, sound-emitting chipsets, misting mechanisms, or adhesives or inserts that can transmit chemical compounds upon contact with skin, in addition to aromatherapy inserts that emit an aromatic compound. The tactile electrodes may emit vibrations or provide haptic feedback for vibrational therapy. The chipsets may emit sound, including music, for sound therapy. The misting mechanisms may emit one or more mists upon activation by a mechanical or electronic trigger. Circuitry to control the medicinal dosing functionality may be incorporated directly into the ornament 120. Alternatively, at least some of the circuitry may be provided in an electronic device, such as a smartphone, that is connected with communication circuitry in the ornament 120 such that the ornament 120 may be controlled remotely.

The ornament (and/or remote) circuitry may include electronic timers, biometric sensors and/or environmental conditional sensors. Such sensors may be capable of detecting and signaling, by sound, vibration or lighting (e.g., LEDs), that one or more specified conditions have been met or exceeded. In some embodiments, the ornament may have the ability to also trigger not only its own dosing capability, but in addition, an adjacent or nearby ornament, if present. The triggering may be via, for example, device-to-device (D2D) communication or may be mediated by an intermediary such as a smartphone. In the latter case, the indication/triggering signal from one ornament may be provided to the intermediary and then to the nearby ornament. The medicinal dosing functionality (e.g., sound, vibration, lighting) may depend on the specified condition that has been met/exceeded. The medicinal dosing functionality and specified conditions may be ornament-independent or may be dependent on the ornament (for ornaments that provide the same medicinal dosing functionality).

With regards to wellness insights capabilities, the ornament may contain the functionality to leverage existing non-intrusive biosensing technologies, such as but not limited to, temperature reading, pulse reading, and CO₂ reading. The ornament may have a memory in which the sensor readings may be stored for later transmission through Bluetooth (BT), near field communication (NFC) or other signaling technologies (e.g., WiFi or 3GPP technologies). The sensor readings may be provided in response to an event occurring, e.g., periodically to another electronic device, in response to a request for the sensor readings or in response to one or more sensor readings meeting/exceeding one or more thresholds. Alternatively, or in addition, such readings can be displayed immediately or when activated through a visual interface on the ornament itself. Note that the experience may not be limited to a single apparatus within the ornament hosting all qualities; the ornament may be made up of several solid adjacent components or adjacent ornaments can interact with each other, by means of BT, NFC and/or signaling technologies.

In some embodiments, an active ornament may have a pattern such as that shown in FIG. 1 on a surface facing the user and environment. The active ornament may contain an inner recess in which the functional insert is placed and may be replaced if desired. In some embodiments, the active ornament may have one or more catches, clasps, and/or other fastening mechanisms to permit the active ornament to be opened and closed, e.g., by sliding the cover of the ornament around a support point or flipping open the cover of the ornament on a hinge, thereby allowing the insert to be introduced to and removed from the recess. The recess and insert may be designed to provide minimal tolerance such that the insert does not move around laterally (towards the edges) and/or vertically (towards the faces) within the recess when the shirt is being worn. In some embodiments, a separate retention mechanism may be formed within the recess to provide vertical and/or lateral compression force to limit movement of the insert within the recess. Other active ornaments, e.g., ones that contain a different functional insert such as electronics may also be used.

The aromatic compound may be one or more essential oils, for example, from flowers or plants. The healing crystal may be formed, for example, from one or more semiprecious stones and crystals such as quartz, amethyst or opals. If the ornament comprises at least one aromatic compound, in some embodiments, one or more of the aromatic compounds may be continuously active (i.e., open and emitting an aroma continuously without manual intervention). In other embodiments, one or more of the aromatic compounds may be activated manually while on the garment to allow the user more control of the aroma. For example, a slide or other mechanical or electrical activator may be disposed on the body 122 of the ornament 120 to allow the wearer to adjust one or more openings in the body 122 to allow different amounts of aroma to persist.

In some embodiments, the attachment mechanism 126 may be disposed on the support 124 at opposing sides of the body 122. The attachment mechanism 126 may be connected to the anchor segment 110 using a button/snap combination. In such embodiments, as shown, a snap button 114a, 114b may be, or may be formed in, each anchor segment piece 112a, 112b. The body 122 of the ornament 120 may have loops formed to retain the support 124, which may be fabric or other flexible or semi-flexible material extending therethrough. The attachment mechanism 126 may be a snap, with which the button 114a, 114b may be engaged.

Other combined attachment mechanisms may be used to attach the anchor segment piece 112a, 112b and the ornament 120, such as Velcro or a slit/button combination (e.g., a slit may be formed in each anchor segment piece 112a, 112b and a button may be formed on opposing portions of the support 124, or vice-versa). Each attachment mechanism 126 may be proximate to an end of the support 124 or may be disposed, as shown, on the side of the body 122 closer to the middle of the support 124 on that side. The support 124 may be of a length such that the support 124 is substantially taught when the snap and buttons engage. In other embodiments, the ornament 120 may be replaceably attached to the anchor segment 110 directly using connectors on both the ornament 120 and each of the anchor segment pieces 112a, 112b, such as hasps or hooks and catches.

Although only one ornament and anchor segment, in some embodiments, multiple anchor segments and/or ornaments may be disposed in essentially the same location on the garment. These multiple ornaments may contain the same components or may contain different components. For example, multiple aromatherapy ornaments, multiple crystal ornaments or geodes multiple medicinal dosages mechanism, or multiple technologies for wellness insights, any combination of multiple components may be disposed on the same anchor or at least one aromatherapy ornament or at least one crystal ornament or at least one medical dosage mechanism or at least one wellness technologies may be disposed on the same anchor segment. In this case, the multiple ornaments may be designed to be disposed next to each other (laterally stacked) or on top of each other (depth stacked). The anchor segment may have multiple pairs of anchor segment pieces and thus button/snap for each ornament (such as for laterally stacked ornaments), with each ornament using a different pair of anchor segment pieces. Alternatively, one or more single anchor segment pieces (and thus a single button/snap) of the multiple pair of anchor segment pieces may be used for multiple ornaments. In another embodiment, the anchor segment may have a single pair of anchor segment pieces, and thus a single button/snap, that is used by all ornaments (such as for depth stacked ornaments).

In further embodiments, the multiple anchor segments and/or ornaments may be disposed in different locations on the garment. For example, rather than being disposed only on a single shoulder portion_ or sleeve, one or more anchor segments and/or ornaments (as above) may each be disposed on opposite shirt shoulder portions or sleeves and/or on other portions of a shirt. Thus, in some embodiments, one or more anchor segments and/or ornaments may be disposed on a shoulder portion or sleeve while one or more other anchor segments and/or ornaments may be disposed at a left breast/heart area of the main portion of the shirt, at a bottom of the back of the shirt (over lower portion of the back), and/or vertically along the back of the shirt from essentially the top to the bottom of the shirt (e.g., along a spine) inclusive of along the sleeve as far as reaching the wrist. The anchor segments and/or ornaments may be disposed on the internal portion of the shirt facing the wearer or the external portion of the shirt facing the environment, with different anchor segments and/or ornaments in the same and/or different locations being able to be disposed on the internal and/or external portion of the shirt.

Moreover, in various embodiments, the orientation of the anchor segment and/or ornament may be different than that shown in FIGS. 1 and 2. In particular, the orientation shown is essentially horizontal relative to the shoulder portion or sleeve length. However, in some embodiments, such as those when the anchor segment/ornament are disposed on straps of a tank top or when the anchor segment and/or ornament is along a spine, the anchor segment/ornament may be disposed in a different orientation, such as a vertical orientation.

In addition, the shape of anchor segments and/or ornaments may be different from that shown. In FIGS. 1 and 2, the anchor segment is rectangular while the ornaments are circular (aromatherapy) or substantially circular (crystal). However, other shapes may be used, including rectangular, triangular, ovular, or other regular or irregular geometric shapes.

Note that although the garment is described as a shirt, in other embodiments, similar ornaments may be added to other clothing articles, such as pants, skirts, neckwear, gloves, socks, shoes or headgear, among others. In such embodiments, the garment may for example be designed to cover at least a portion of a center portion of an individual wearing the garment, such as pants covering a lower range of the center portion of the individual.

In other embodiments, the garment may be used for pets and other animals. The placement of the anchor segment/ornament may be dependent on the animal type.

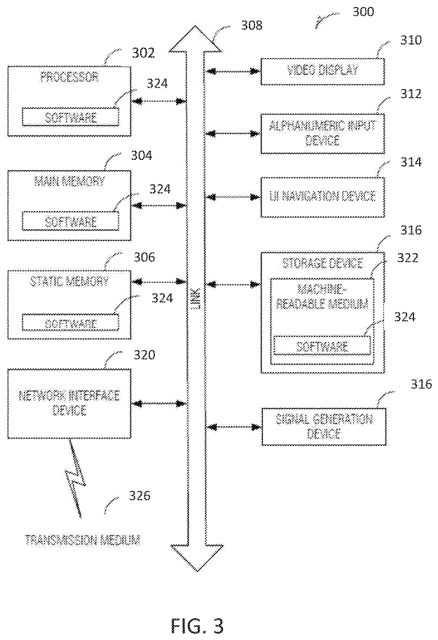

FIG. 3 illustrates electronic components of an ornament in accordance with some embodiments. The ornament 300 may include a communication device that may be any machine capable of executing instructions (sequential or otherwise) that specify actions to be taken by that machine. Examples, as described herein, may include, or may operate on, logic or a number of components, modules, or mechanisms. Modules and components are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” (and “component”) is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The communication device 300 may include a hardware processor 302 (e.g., a central processing unit (CPU), a GPU, a hardware processor core, or any combination thereof), a main memory 304 and a static memory 306, some or all of which may communicate with each other via an interlink (e.g., bus) 308. The main memory 304 may contain any or all of removable storage and non-removable storage, volatile memory or non-volatile memory. The communication device 300 may further include a display unit 310 such as a video display, an alphanumeric input device 312 (e.g., a keyboard), and a user interface (UI) navigation device 314. In an example, the display unit 310, input device 312 and UI navigation device 314 may be a touch screen display. The communication device 300 may additionally include a storage device (e.g., drive unit) 316, a signal generation device 318 (e.g., a speaker), a network interface device 320, and one or more sensors, such as a global positioning system (GPS) sensor, compass, accelerometer, or other sensor. The communication device 300 may further include an output controller, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 316 may include a non-transitory machine readable medium 322 (hereinafter simply referred to as machine readable medium) on which is stored one or more sets of data structures or instructions 324 (e.g., software) embodying or utilized by any one or more of the techniques or functions described herein. The instructions 324 may also reside, successfully or at least partially, within the main memory 304, within static memory 306, and/or within the hardware processor 302 during execution thereof by the communication device 300. While the machine readable medium 322 is illustrated as a single medium, the term “machine readable medium” may include a single medium or multiple media (e.g., a centralized or distributed database, and/or associated caches and servers) configured to store the one or more instructions 324.

The term “machine readable medium” may include any medium that is capable of storing, encoding, or carrying instructions for execution by the communication device 300 and that cause the communication device 300 to perform any one or more of the techniques herein, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks.

The instructions 324 may further be transmitted or received over a communications network using a transmission medium 326 via the network interface device 320 utilizing any one of a number of transfer protocols (e.g., frame relay, Internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks. Communications over the networks may include one or more different protocols, such as Institute of Electrical and Electronics Engineers (IEEE) 802.11 family of standards known as Wi-Fi, IEEE 802.16 family of standards known as WiMax, IEEE 802.15.4 family of standards, a Long Term Evolution (LTE) family of standards, a Universal Mobile Telecommunications System (UMTS) family of standards, peer-to-peer (P2P) networks, a NG/NR standards among others. In an example, the network interface device 320 may include one or more physical jacks (e.g., Ethernet, coaxial, or phone jacks) or one or more antennas to connect to the transmission medium 326.

From the foregoing description, one skilled in the art can easily ascertain the essential characteristics of this invention and, without departing from the spirit and scope thereof, can make various changes and modifications of the invention to adapt it to various usages and conditions.

Although an aspect has been described with reference to specific example aspects, it will be evident that various modifications and changes may be made to these aspects without departing from the broader scope of the present disclosure. Accordingly, the specification and drawings are to be regarded in an illustrative rather than a restrictive sense. The accompanying drawings that form a part hereof show, by way of illustration, and not of limitation, specific aspects in which the subject matter may be practiced. The aspects illustrated are described in sufficient detail to enable those skilled in the art to practice the teachings disclosed herein. Other aspects may be utilized and derived therefrom, such that structural and logical substitutions and changes may be made without departing from the scope of this disclosure. This Detailed Description, therefore, is not to be taken in a limiting sense, and the scope of various aspects is defined only by the appended claims, along with the full range of equivalents to which such claims are entitled.

The Abstract of the Disclosure is provided to comply with 37 § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single aspect for the purpose of streamlining the disclosure. This method of disclosure is not to be interpreted as reflecting an intention that the claimed aspects require more features than are expressly recited in each claim. Rather, as the following claims reflect, inventive subject matter lies in less than all features of a single disclosed aspect. Thus, the following claims are hereby incorporated into the Detailed Description, with each claim standing on its own as a separate aspect.

What is claimed is:

1. A garment comprising:
a garment body configured to cover at least a portion of a center portion of an individual wearing the garment, the garment body comprising a plurality of openings configured to receive different appendages of the individual passing therethrough;
an anchor segment attached to the garment body such that the anchor segment lies substantially flat against the garment body on which the anchor segment is disposed; and
an ornament replaceably attached to the anchor segment, the ornament comprising at least one of an aromatic compound, a crystal, a medical loser or a wellness insight technology.

2. The garment of claim 1, wherein:
the garment is a shirt, and
the garment body comprises shoulder portions of the shirt, the anchor segment disposed on one of the shoulder portions.

3. The garment of claim 2, wherein the anchor segment is disposed on only one of the shoulder portions.

4. The garment of claim 1, wherein if the ornament comprises the aromatic compound, the aromatic compound is activated manually while on the garment.

5. The garment of claim 1, wherein the anchor segment is permanently connected to the garment body.

6. The garment of claim 1, wherein the ornament comprises:
an ornament body, and
ornament connectors through which the ornament is attached to the anchor segment, the ornament connectors configured to engage with anchor segment connectors on the anchor segment, the ornament connectors disposed on opposite sides of the ornament body.

7. The garment of claim 6, wherein:
the ornament further comprises a support on which the ornament connectors are disposed, and
the ornament body comprises loops through which the support passes to teach the ornament body to the support.

8. The garment of claim 7, wherein the ornament body comprises a surface opposing a surface facing the garment body, the surface having openings through which scent is emitted if the ornament comprises the aromatic compound.

9. The garment of claim 6, wherein:
the anchor segment comprises K2 buttons disposed on opposing sides of the ornament body,
the ornament body is attached to a piece of fabric, and
the piece of fabric has snaps on opposing sides of the ornament body that attach the ornament to the anchor segment via the K2 buttons.

10. The garment of claim 8, wherein:
the anchor segment comprises separate anchor segment pieces disposed on opposite sides of the ornament body,
the ornament body is attached to a piece of fabric,
each anchor segment piece comprises one of a button or snap, and
the piece of fabric has another of the button or snap on opposing ends that attach to the anchor segment pieces through the one of the button or snap.

11. The garment of claim 1, wherein the ornament comprises:
an ornament body comprising a recess therein configured to retain an insert therein, and
a fastening mechanism that permits the ornament body to be opened and closed allowing the insert to be inserted into and removed from the recess.

12. The garment of claim 11, wherein if the ornament comprises the aromatic compound, the insert comprises the aromatic compound.

13. A shirt comprising:
a body configured to cover at least a portion of a torso of an individual wearing the garment, the garment body comprising shoulder portions;
sleeves connected to the garment body;
an anchor segment permanently attached to at least one of the shoulder portions or sleeves; and
an ornament replaceably attached to the anchor segment, the ornament comprising at least one of an aromatic compound or a crystal.

14. The shirt of claim 13, wherein the ornament comprises:
an ornament body, and
ornament connectors through which the ornament is attached to the anchor segment, the ornament connectors configured to engage with anchor segment connectors on the anchor segment, the ornament connectors disposed on opposite sides of the ornament body.

15. The shirt of claim 14, wherein:
the ornament further comprises a support on which the ornament connectors are disposed, and
the ornament body comprises loops through which the support passes to attach the ornament body to the support.

16. The shirt of claim 15, wherein:
the support comprises a piece of fabric,
each anchor segment piece comprises one of a button or snap, and
the piece of fabric has another of the button or snap on opposing ends that attach to the anchor segment pieces via the one of the button or snap, the ornament connectors comprising the other of the button or snap.

17. The shirt of claim 14, wherein the ornament body comprises:
a recess therein configured to retain the at least one of the aromatic compound or crystal therein, and
a fastening mechanism that permits the ornament body to be opened and closed, allowing the at least one of the aromatic compound or crystal to be inserted into and removed from the recess.

18. The shirt of claim 17, wherein the ornament body further comprises a surface opposing a surface facing the garment body, the surface having openings through which scent is emitted if the at least one of the aromatic compound or crystal comprises the aromatic compound.

19. A method of fabricating a shirt, the method comprising:
selecting an ornament comprising at least one of an aromatic compound or a crystal; and
attaching the ornament to a shoulder portion of the shirt via anchor segment pieces of an anchor segment.

20. The method of claim 19, further comprising:
permanently attaching the anchor segment pieces to the sleeve before attaching each anchor segment piece to an opposite side of the ornament,
wherein the ornament is attached to a piece of fabric,
each anchor segment piece comprises one of a button or snap, and
the piece of fabric has another of the button or snap on opposing ends that attach to the anchor segment pieces via the one of the button or snap.
